(19) United States
(12) Patent Application Publication
(10) Pub. No.: US 2012/0150111 A1
(43) Pub. Date: Jun. 14, 2012

(54) Passive Enteral Venting System

(76) Inventors: Adrienne A. Hershey, Cumming, GA (US); Alison S. Bagwell, Alpharetta, GA (US); Donald J. McMichael, Roswell, GA (US)

(21) Appl. No.: 12/967,681

(22) Filed: Dec. 14, 2010

(51) Int. Cl. A61M 25/00

(52) U.S. Cl. 604/122

(57) ABSTRACT

There is provided a venting system for enteral feeding. The system has a tube in fluid communication with an enteral feeding site on a patient. The tube receives gas and liquid from the patient as enteral pressure within the patient increases. The tube has a vent that relieves enteral gas pressure from the patient and the tube also returns any liquid to the patient. There is also provided an enteral feeding system having a nutrient source in fluid communication with a nutrient pump. The pump discharges to an enteral feeding site on a patient. The system has a vent bag also in fluid communication with the enteral feeding site and the bag receives gas and liquid from the site as enteral pressure increases within the patient. The bag has a vent that relieves enteral gas pressure and allows liquid to be returned to the patient via the pump.

The present disclosure relates to a system for venting in connection with enteral feeding, to reduce gas pressure and discomfort for patients.

Many patient feeding devices employ a gastrostomy feeding tube. One relatively common situation is to provide nutritional solutions or medicines directly into the stomach or intestines. A stoma is formed in the stomach or intestinal wall and a catheter is placed through the stoma. Such catheters generally have a base connector for tubing on the proximal end and a balloon on the distal to keep the catheter in place. Feeding solutions can be injected through the catheter at an enteral feeding site on the patient to provide nutrients directly to the stomach or intestines in a process known as enteral feeding. A variety of different catheters intended for enteral feeding have been developed over the years, including some having a “low profile” base connector relative to the portion of the catheter which sits on a patient's skin, as well as those having the more traditional or non-low profile configuration.

Enteral feeding may be necessary because of a number of causes, one of which is the not uncommon reaction following major surgery in which a patient's stomach function is impaired for a period of time. If the patient has a problem with gastric reflux or vomiting, for example, or if the stomach is not adequate for the patient's digestive process requirements, another feeding mode must be chosen. In addition to the need to supply or supplement the body with a certain level of nutrients and the like following surgery as well as in other instances of impaired or limited gastric functionality, a further issue is that an unfed gut can become a source of bacteria that gets into the bloodstream.

In enteral feeding, after the placement of the feeding head and catheter in the patient's body, nutrients are pumped from a bag into the catheter and into the stomach or intestine. The nutrient bag may be hung above the bed of the patient and the feeding pump placed nearby.

While the problems noted above can be solved by the introduction of nutrients through an enteral feeding device tube properly inserted through the patient's abdominal wall, a problem of pressure buildup in the intestines and/or stomach is quite common. This pressure buildup is the result of normal digestive processes but can be very painful, especially for pediatric patients. Typically the body relieves such excess gastric pressure through expulsion of accumulated gas or liquid through a burping response. However, in a patient undergoing enteral feeding in which fluid nutrients are being continually fed to the gastrointestinal tract, upward expulsion of gastric reflux materials is highly undesirable. More importantly, reflux of gas or liquid through the enteral feeding tube cannot occur.

Though gastric reflux pressure created by even limited episodes of stomach movement may exceed several feet of water, such reflux pressure is inadequate to overcome the greater forward fluid pressure present within the enteral feeding tube. This greater fluid pressure is developed because the height of the column of fluid nutrient in the enteral feeding system usually stands well above the level of the patient's stomach. Fluid pressure is further increased through the use of the enteral feeding pump. In addition, tube set clamps along the administration tubing also prevent reflux of excessive gastric gas or liquid through the enteral feeding tube.

Because gastric reflux pressure cannot overcome the greater forward fluid pressure within the enteral feeding tube, reflux materials may be expelled upward from the stomach through the esophagus and are expressed out of the mouth, where the enteral feeding tube is orally intubated, or through the nasal passages, where naso-pharyngeal intubation has been utilized. In the latter, it is possible for the patient to inhale the reflux materials into the lungs with possible risk of aspiration pneumonia. The problem of relief of gastric reflux pressure is most accute in neonates, infants and small children in which gastric pressure may rapidly accumulate through periodic episodes of crying and because such patients have yet to develop control over the burping response as a means of gastric pressure relief. However, it is not unusual for adult patients undergoing enteral feeding to experience occasional difficulties with gastric reflux pressure relief.

A number of possible solutions have been suggested to relieve pressure in the digestive tract. One widely used option is the Farrell valve available from Corpak® MedSystems of Wheeling Ill., described in U.S. Pat. No. 6,482,170. This system was developed to permit relief of gastric reflux pressure through the enteral feeding tube to avoid uncontrolled upward expulsion of reflux materials through the burping response. The Farrell valve uses a “Y” type connector that is placed in the feeding line between the feeding pump and the patient. An additional line is connected to the “Y” and terminates at a second or vent bag hanging at the same height as the nutrient bag. The vent bag is vented to the atmosphere in order to allow gas to escape the system. Any liquid that is carried along with the gas to the vent bag returns to the patient via the “Y” connector. The “Y” connector is located below the level of the patient's stomach in order to keep at least a small amount of nutrient in the tubing line leading to the vent bag. The small amount of nutrient prevents air from being introduced to the stomach from the vent bag.

While the system of the '170 patent performs satisfactorily, the reflux liquid that is carried to the vent bag with the pressure causing gas usually returns to the patient as a single large dose on a periodic basis, rather than being metered and mixed with the fresh nutrient formulation. This may cause irritation of the patient's enteral feeding site or increased gas production and pain.

There remains a need for a system that can relieve excess pressure from a patient's stomach, return the entrained vented reflux liquid to the patient, and do it in a manner that is more uniform or that may be controlled by the patient. In this manner the patient will receive a more uniform feeding formulation or one of his choosing.

### SUMMARY

The present disclosure describes a venting system for enteral feeding. The system has a tube in fluid communication with an enteral feeding site on a patient. The tube receives gas and liquid from the patient as enteral pressure within the patient increases. The tube has a vent that relieves enteral gas pressure from the patient and the tube also returns any liquid to the patient. The tube may be a vertical cylinder that hangs above the patient, desirably near the nutrient bag and may be a dual lumen tubing.

This disclosure also describes an enteral feeding system having a nutrient source in fluid communication with a nutrient pump. The pump discharges to an enteral feeding site on a patient. The system has a vent bag also in fluid communication with the enteral feeding site and the bag receives gas and liquid from the site as enteral pressure increases within the patient. The bag has a vent that relieves enteral gas pressure and allows liquid to be returned to the patient via the pump.

The venting system may have a valve that allows fluid flow to the pump from either the nutrient source or the vent bag, allowing greater control for the patient to determine the feeding source. The nutrient source and vent bag may be separate bags or may be a single divided bag with the nutrients separated from the gas and liquid on the vent bag side.

The vent may be a slit or hole in the tube or bag that is covered with a nonwoven fabric that is a microporous membrane material. The vent may desirably relieve at a pressure of between 1.4 and 5.5 kPa or more desirably at a pressure of about 3.48 kPa.

Other objects, advantages and applications of the present disclosure will be made clear by the following detailed description of a preferred embodiment of the disclosure and the accompanying drawings wherein reference numerals refer to like or equivalent structures.

### BRIEF DESCRIPTION OF THE DRAWINGS

FIG. 1 is a perspective view of one embodiment of an enteral feeding system having a nutrient bag, a nutrient pump and a vertical tube vent.

FIG. 2 is a perspective view of one embodiment of an enteral feeding system having a double bag for feeding and for venting and a nutrient pump.

FIG. 3 is a bag arrangement having a nutrient portion and a reflux liquid portion with a vent.

FIG. 4 is a bag arrangement having a separate nutrient and venting vent bag with a check valve connection between the two to allow liquid to exit either bag but not flow between the bags.

FIG. 5 is a perspective view of another embodiment of an enteral feeding system having a double lumen nutrient pump discharge line with a vent from the second lumen of the double lumen line.

### DETAILED DESCRIPTION

Reference will now be made to the drawings in which the various elements of the present disclosure will be given numeral designations and in which the disclosure will be discussed so as to enable one skilled in the art to make and use the disclosure. It is to be understood that the following description is only exemplary of the principles of the present disclosure, and should not be viewed as narrowing the pending claims. Those skilled in the art will appreciate that aspects of the various embodiments discussed may be interchanged and modified without departing from the scope and spirit of the disclosure.

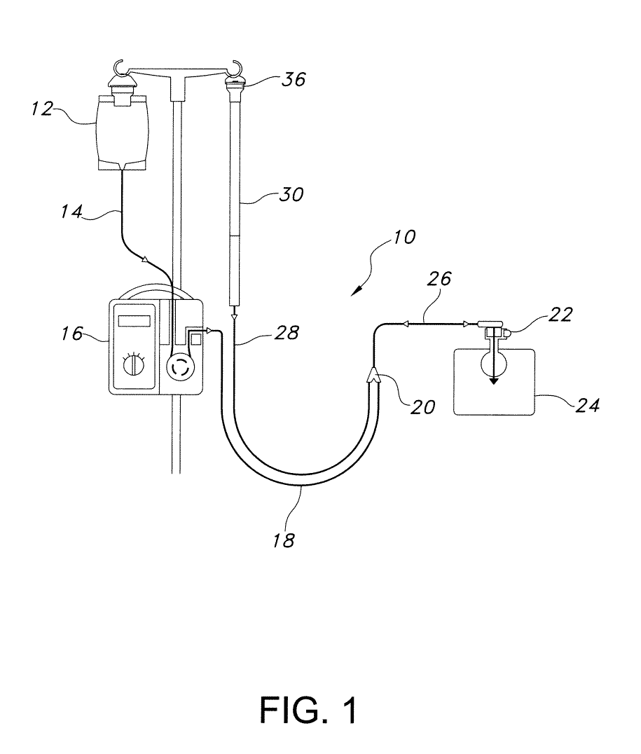

Turning to the drawings, FIG. 1 illustrates an embodiment of the disclosed vent system 10. The device 10 has a nutrient bag 12 containing a feeding formula for the patient 24. The nutrient formula is feed from the bag 12 to the nutrient pump 16 via tubing 14 and is discharged from the pump 16 to the enteral feeding site on the patient, again through tubing 18. A “Y” connector 20 is placed in the pump discharge line 18, i.e., between the pump 16 and the patient 24. An additional length of tubing 28 is connected between the “Y” connector 20 and the vent tube 30. The tube has a vent 36 on its upper most end for the relieving of gas pressure. The patient 24 is equipped with a conventional enteral feeding base connector 22 (including a catheter and balloon as discussed above) for connection of the feeding tubing 26.

The vent tube 30 may be made from plastics like polyethylene and polypropylene, polyamides and other common polymeric materials. The tube 30 may desirably be between 1 and 5 cm in diameter and between 10 and 50 cm in length. The vent tube is desirably hung above the patient in a manner similar to the nutrient bag as shown in FIG. 1. This provides the gravity assist necessary to return liquids to the patient after the gas pressure has been relieved by the vent 36. The vent 36 is made from a material that allows gas to pass through it but does not allow liquid to pass through. The materials from which the vent is made will be discussed in greater detail below but are generally hydrophobic materials like nonwoven fabrics.

In regard to the proper nutrient feeding formulation, both formula characteristics and patient-specific factors need to be considered. Formula variables include: digestibility/availability of the nutrients, nutritional adequacy, viscosity, osmolality, ease of use, and cost. Patient variables include: nutritional status and requirements, electrolyte balance, digestive and absorptive capacity, disease state, renal function, medical or drug therapy, and possible routes available for administration. Adult enteral formula products fall into one of the following categories: general use, high nitrogen, high nitrogen and high calorie, fiber enriched, semi-elemental, fat modified, and specialty. Medical professionals such as dietitians are typically available to assist with formula selection, depending on the location. While the proper selection of nutrient formula for enteral feeding is very important for each individual patient, the myriad of products available makes an exhaustive discussion herein untenable. Suffice to note that the nutrient formulation may be the cause of many different patient complaints, including excessive abdominal pressure, and though the disclosed system can help alleviate pressure, the formulation should also be investigated as a possible cause.

The enteral feeding pump 16 is an electronic medical device that controls the timing and amount of nutrition delivered to a patient during enteral feeding. The enteral feeding pump ensures that the right amount of liquid is administered to the body over the course of a day. The amount of nutrition desired to be fed is entered into the electronic enteral feeding pump, which controls the flow of the nutrition so that the patient gets a measured amount of liquid continuously over a 24 hour period.

Patients who are administering enteral nutrition independently may choose to set their enteral feeding pump on a cyclic cycle. This can allow the patient to administer food over an eight-hour period throughout the night, permitting a more normal lifestyle without the pump during the day. Generally, an enteral feeding pump is very accurate, but problems in the electronic mechanisms can cause too much or too little nutrition to be administered to a patient. Many pumps come with safety features to make such an error extremely unlikely. An enteral feeding pump may have a “no single point of failure feature,” so that the pump either has back-up mechanisms if one component fails, or an audible indication that the pump is no longer working. For devices used in stationary feeding settings, they may also come equipped with a battery pack to back up the unit if the normal electrical power has been dislodged.

Enteral feeding pumps are manufactured by a number of different suppliers. One example of a suitable pump system is available from Covidien AG of Mansfield, Mass. under the Kangaroo label.

In use, nutrients are pumped by the nutrient pump 16 from the nutrient bag 12, to the base connector 22 at the enteral feeding site on the patient 24. As excess gas builds up in the patient's stomach, fluid flow will slow and will eventually reverse flow in the nutrient tubing 26, and then go through the “Y” connector 20 and vent tubing 28 to the vent tube 30. The vent tube will then have a mixture of liquid and gas within it. Gas pressure will continue to build until the gas pressure is eventually relieved by the vent 36 and any liquids that were carried with the gas to the vent tube 30 will be returned to the patient via the vent tubing 28, “Y” connector 20 and base tubing 26. The pressure at which the vent is relieved may be varied by the judicious choice of material from which the vent is made. Further discussion of vent materials and relief pressures may be found below.

While the disclosed vent tube 30 is described above as being connected to the “Y” connector 20 via vent tubing 28, it is also possible to connect the vent tube 30 directly to the enteral feeding base connector 22 without the administration of nutrients. In such a case, the vent tube 30 may be connected to the base connector 22 without the balance of the feeding system, i.e. the pump 16 and nutrient bag 12 and all the associated tubing. In this configuration the vent tube 30 may be used to relieve uncomfortable gas pressure even when the patient is not being fed, where the gas pressure increase may be unconnected with the delivery of nutrients to the patient.

In yet another embodiment, the vent tube 30 may be connected to the base connector 22 while simultaneously connecting the balance of the feeding system to the base connector 22 by using a “T” fitting at the base connector 22. Such a connection occurs closer to the patient, which may facilitate the reintroduction of reflux liquid but entails the use of two tubes in close proximity of the patient.

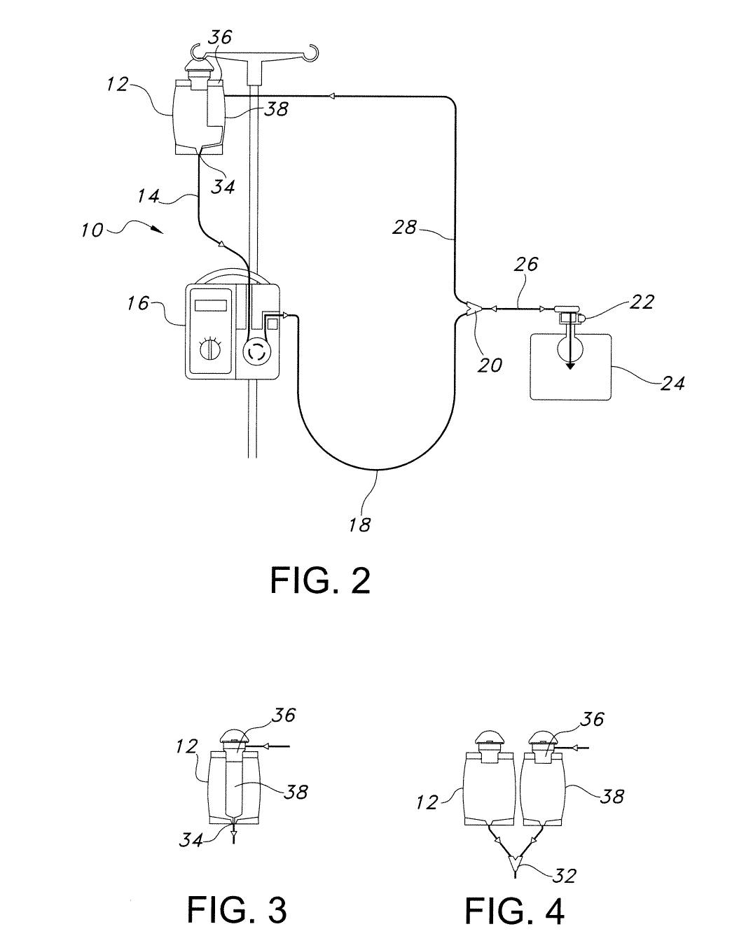

Another venting system embodiment is shown in FIG. 2. The device 10 has a nutrient bag 12 containing a feeding formula for the patient 24. The nutrient formula is feed from the bag 12 to the nutrient pump 16 via tubing 14 and is discharged from the pump 16 to the enteral feeding site on the patient, again through tubing 18. A “Y” connector 20 is placed in the pump discharge line 18, i.e., between the pump 16 and the patient 24. An additional length of tubing 28 is connected between the “Y” connector 20 and the vent bag 38. The patient 24 is equipped with a conventional enteral feeding base connector 22 (including a catheter and balloon as discussed above) for connection of the feeding tubing 26. The nutrient bag 12 has a vent bag 38 that in this case is a segregated section of the nutrient bag 12. Such an arrangement makes the setting up of this enteral feeding system faster and simpler than other systems since only one bag need be hung near the patient. The vent bag 38 has a vent 36 on the upper part of the bag through which pressure is relieved.

In use, nutrients are pumped by the nutrient pump 16 from the nutrient bag 12, to the base connector 22 at the enteral feeding site on the patient 24. As excess gas builds up in the patient's stomach, fluid flow will slow and will eventually back up in the nutrient tubing 26, through the “Y” connector 20 and vent tubing 28 to the vent bag 38. Gas pressure will be relieved by the vent 36 on the vent bag 38 and any liquids that were carried with the gas to the vent bag 38 will be returned to the patient. The nutrient bag 12 and vent bag 38 may be manufactured as one bag having a divider as shown in FIG. 2.

The vent bag (or nutrient bag) has a valve 34 at the bottom that allows fluid flow to the pump 16 from either the nutrient bag 12 portion or the vent bag 38 portion, but does not allow fluid flow between the two. This is an advantage over other systems because the user may determine from which source the patient is fed. Allowing greater control for the user (or patient) to determine the feeding source increases the flexibility and utility of the system.

FIG. 3 shows a variation of the nutrient bag 12/vent bag 38 combination where the vent bag 38 is molded into the body of the nutrient bag 12 in the central area. FIG. 4 shows the nutrient bag 12 and vent bag 38 as separate bags with a one way connection 32 between them that allows fluid flow from either bag but does not allow fluid to flow from one bag to the other. In both of these arrangements the user may determine from which source the patient is fed, giving more control to fine tune the patient's nutrition to the user.

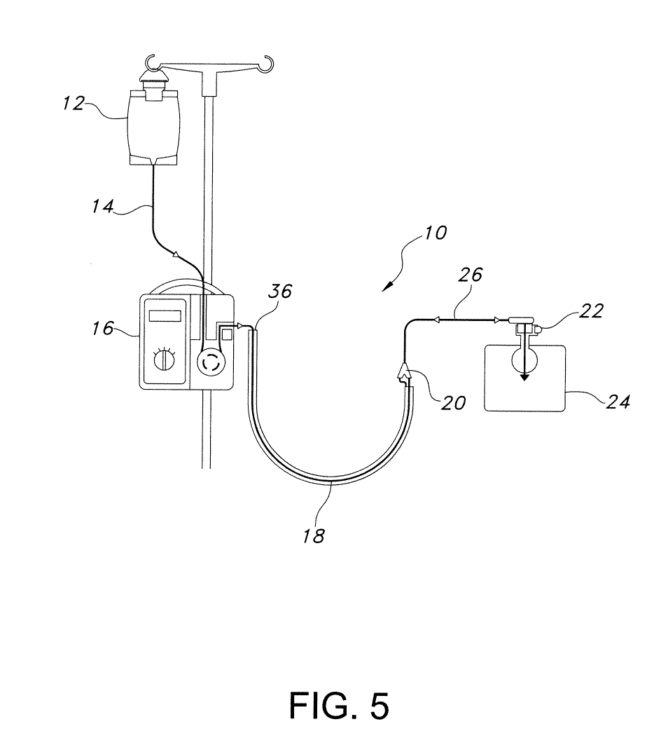

In still another embodiment, the device 10 has a nutrient bag 12 containing a feeding formula for the patient 24. The nutrient formula is feed from the bag 12 to the nutrient pump 16 via tubing 14 and is discharged from the pump 16 to the enteral feeding site on the patient, again through tubing 18. The discharge tubing 18 has a first (desirably central) lumen through which the nutrient formula is pumped to the patient 24 (FIG. 5). A second, desirably larger lumen is provided for the venting of the enteral pressure from the patient. A “Y” connector 20 is placed in the pump discharge line 18, i.e., between the pump 16 and the patient 24. When a high pressure occurs, gas and some entrained reflux liquid are vented into the second lumen of the tubing 18. A vent 36 at the proximal end of the tubing 18 allows gas to escape from the second lumen of the tubing 18 but does not allow liquid to escape. The reflux liquid returns from the second lumen to the patient through the “Y” connector 20. Alternatively, the reflux liquid could be returned to the patient via a valve and tubing (not shown) that allows fluid flow to the pump 16 from either the nutrient bag 12 or the second lumen, but does not allow fluid flow between the nutrient bag and second lumen. (This same alternative arrangement could be used for the embodiment of FIG. 1 as well).

The vent 36 is adapted to relieve pressure from the vent bag 38 at a pressure of about 0.5 pounds per square inch (psi) (3.48 kilopascal) (kPa), a pressure at which it has been documented that pain from stomach gas pressure starts to be felt. Other vent pressure relief points may be chosen depending on medical factors related to a specific patient, so the relief pressure may be from 0.2 to 0.8 psi (1.4 to 5.5 kPa). The vent pressure may be chosen by varying the type or number of layers of the materials from which the vent is made. A highly porous vent material, for example, will vent before a less porous material will vent. Similarly, a single layer vent material will vent before a multi-layer vent made from the same material will vent.

The vent 36 desirably comprises a membrane that may be a nonwoven material that is placed over a slit or hole in the vent bag 38 or tube 30. The membrane material may be glued to the edges of the slit in the bag using any suitable adhesive or may be heat sealed (welded) or attached in other ways known to those skilled in the art. Heat sealing, for example, uses heat, pressure and dwell time to thermally bond thermoplastic materials together. Heat sealing devices generally have a press with a set of jaws that open (vertically), into which the materials to be bonded are placed. The jaws are heated by, for example, electric resistance heating and the temperature of each may be controlled separately. The pressure at which the jaws come together may also be adjusted for optimal bonding. Lastly, the time for which the jaws are together (the “dwell” or “hover” time) may also be adjusted. A dwell time of zero indicates that the jaws were brought together for an instant and immediately moved apart, i.e., they were not held together.

The size of the vent 36 may vary slightly depending on the porosity of the membrane material used. In the embodiment shown, the vent is less than about 5 square centimeters in size. Vent sizes as low as 1 square cm and as great as 10 square cm can be used through extremely large vent areas may comprise the structural integrity of the vent bag or tube somewhat and make the bag difficult to handle.

The vent membrane materials are generally hydrophobic in order to retain liquid within the vent bag while allowing gas to pass through it. These materials are also hydrophobic because it is important that the membrane material not become degraded by contact with any liquid present in the bag.

Suitable nonwoven membrane materials include microporous materials such as Millipore® DVSP vent membrane using Surevent® PVDF membrane material with a 0.65 micron pore size, Millipore® BVSP vent membrane using Surevent® PVDF membrane material with a 1 micron pore size, Millipore® BVSPW vent membrane using Surevent® PVDF membrane material with a 1 micron pore size, Millipore® DOHP vent membrane using Surevent® UPE membrane material with a 0.65 micron pore size, Millipore® UPBP vent membrane using Surevent® UPE membrane material with a 1 micron pore size, Millipore® BPTFEPP vent membrane using Surevent® PTFE membrane material with a 1 micron pore size, Millipore® BPTFEPE vent membrane using Surevent® PTFE membrane material with a 1 micron pore size, Gore® MMT 332 vent membrane with expanded PTFE membrane material with a 1 micron pore size, Porex® vent membrane with Mupor® PTFE membrane material with a 4 micron pore size, Porex® vent membrane with X-7744PE membrane material with a 10 micron pore size, Pall® vent membrane with Versapor® 800R (acrylic) membrane material with a 0.8 micron pore size and Pall® vent membrane with Versapor® 500R (acrylic) membrane material with a 5 micron pore size.

Millipore® materials are available from Millipore® Corporation of Billerica, Mass. Gore® materials are available from WL Gore® & Associates of Newark, Del. Porex® materials are available from Porex® Corporation of Fairburn, Ga. Pall® materials are available from Pall® Corporation of Port Washington, N.Y.

As used herein and in the claims, the term “comprising” is inclusive or open-ended and does not exclude additional unrecited elements, compositional components, or method steps.

While the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A venting system for enteral feeding comprising a tube in fluid communication with an enteral feeding site on a patient, said tube receiving gas and liquid from said patient, said tube having a vent that relieves enteral gas pressure from said patient and wherein said tube returns any liquid to said patient.

2. The venting system of claim 1 wherein said vent relieves at a pressure of between 1.4 and 5.5 kPa.

3. The venting system of claim 1 wherein said vent relieves at a pressure of about 3.48 kPa.

4. The venting system of claim 1 wherein said vent comprises a slit or hole in said tube and said slit or hole is covered with a nonwoven fabric that is a microporous membrane material.

5. The venting system of claim 1 wherein said tube comprises a double lumen tubing wherein one of said lumens delivers nutrients to said patient.

6. The venting system of claim 1 wherein said liquid is returned to said patient via a pump.

7. An enteral feeding system comprising a nutrient source in fluid communication with a nutrient pump that discharges to an enteral feeding site on a patient; and,
a vent bag also in fluid communication with said enteral feeding site, said bag receiving gas and liquid from said site, said bag having a vent that relieves enteral gas pressure from said patient and allows liquid to be returned to said patient by said pump.

8. The venting system of claim 7 further comprising a valve that allows fluid flow to the pump from either the nutrient source or the vent bag, allowing greater control for the patient to determine the feeding source.

9. The venting system of claim 7 wherein said bag is a single divided bag containing nutrients separated from vent gas and liquid.

10. The venting system of claim 7 wherein said nutrient bag and said vent bag are separate bags.

11. The venting system of claim 7 wherein said vent comprises a slit or hole in said bag and said slit or hole is covered with a nonwoven fabric that is a microporous membrane material.

12. The venting system of claim 7 wherein said vent relieves at a pressure of between 1.4 and 5.5 kPa.

13. The venting system of claim 7 wherein said vent relieves at a pressure of about 3.48 kPa.
